(19) United States
(12) Patent Application Publication
(10) Pub. No.: US 2023/0035038 A1
(43) Pub. Date: Feb. 2, 2023

(54) PLANT-BASED COMPOSITION IN A FORM SUITABLE FOR TOPICAL ADMINISTRATION AND ASSOCIATED PRODUCTION METHOD

(71) Applicant: MENELIK, Maisons-Laffitte (FR)

(72) Inventor: Alain CASMEZE, Paris (FR)

(73) Assignee: MENELIK, Maisons-Laffitte (FR)

(21) Appl. No.: 17/759,122

(22) PCT Filed: Jan. 19, 2021
(86) PCT No.: PCT/EP2021/050982

§ 371 Date: Jul. 20, 2022

(30) Foreign Application Priority Data
Jan. 20, 2020 (FR) FR2000515

(51) Int. Cl. A61K 36/73; A61K 36/288; A61K 9/00; A61K 36/53; A61K 36/282; A61K 36/76; A61K 36/68; A61K 36/882; A61K 36/515; A61K 36/25; A61P 9/00

(52) U.S. Cl. CPC A61K 36/73; A61K 36/288; A61K 9/0014; A61K 36/53; A61K 36/282; A61K 36/76; A61K 36/68; A61K 36/882; A61K 36/515; A61K 36/25; A61P 9/00

(57) ABSTRACT

A composition in a form suitable for topical administration has, in a cosmetically and/or pharmaceutically acceptable vehicle, at least one nettle (Urtica) extract, and can include a mixture of at least five plant extracts. Methods to manufacture such a composition are also disclosed. Such a composition is particularly useful for the prevention, treatment, relief and alleviation of the symptoms of haemorrhoidal disease as well as aiding healing, and soothing burns.

The present invention relates to a composition in a form suitable for topical herbal administration, as well as to a process for its manufacture. The invention also relates to such a composition for use in the treatment or prevention of symptoms in dermatological or gastroenterological conditions, particularly proctological conditions such as haemorrhoidal disease.

Haemorrhoids are normal vascular formations around and within the anus and rectum, which are present in all individuals and are painless. Internal haemorrhoids are located within the anal canal under the lining of the intestinal mucosa. It is a complex and peculiar system of vessel organisation: it has venous sacs covered by arterioles, the blood content of which can vary very rapidly. The volume of the haemorrhoidal columns accounts for 10 to 20% of the anal seal. External haemorrhoids are located under the skin margin of the anus (anal margin). Both systems have vascular connections under the anal mucosa.

These vascular formations are circumferential but concentrate in haemorrhoidal “columns” depending on the number of arterial pedicles that feed them. These columns are usually three in number: right anterior, right posterior and left lateral. They also act as a shock absorbing pad during rectal evacuation. They maintain their anatomical position (especially during bowel movements) thanks to elastic fibres from the sphincter system. Thus, in the normal state, the presence of haemorrhoids is not noticeable.

Haemorrhoidal disease is a benign condition with very uncomfortable symptoms. The main symptoms of haemorrhoidal disease are bleeding, pain and swelling. There are generally four grades of anatomical changes during a proctological examination.
Grade 1: Hemorrhoidal symptoms without any externalisation.
Grade 2: Fleeting externalisation during defecation.
Grade 3: Frank exteriorisation on defecation or effort, followed by slow spontaneous reduction or assisted by finger pressure.
Grade 4: permanent externalisation (prolapsed haemorrhoids).

Medical treatment, which is based on regulating intestinal transit by treating constipation, on the administration of drugs with a venous tonic effect and local topicals (creams and suppositories) and instrumental treatment carried out at the surgery (infrared, elastic ligation, etc.) are generally no longer effective when there is a significant prolapse. At this stage, haemorrhoidal surgery is usually indicated depending on the patient's request. Only 10% to 15% of patients require surgical treatment.

Current surgical techniques are based on two different principles: either a haemorrhoidectomy (or Millligan & Morgan technique), i.e. the removal of the internal and external haemorrhoidal columns, or a haemorrhoidopexy (or Longo process) which consists of repositioning and fixing the internal haemorrhoids in the anal canal.

Haemorrhoidopexy is indicated when the prolapse of the internal haemorrhoids is symmetrical over the three main columns or almost circumferential and being a grade 3. It is particularly suitable for patients who have to make efforts during defecation and in whom it is often found on examination with an anoscopy that the prolapsed haemorrhoids are accompanied by an internal prolapse of the mucosa of the lower rectum. The advantage of this procedure is that it maintains the integrity of the anal canal and anal margin with a technique that avoids an operative wound. The Longo process is less painful postoperatively and allows a quick return to normal life.

A haemorrhoidectomy is a procedure that can be performed under any circumstances. It is necessary when only one or two haemorrhoidal columns are pathological and can be removed with little pain after the operation. It is used to treat external haemorrhoidal disease (recurrent thrombosis, tags or residual skin excess). It guarantees the result in the event of permanently externalised hemorrhoids (grade 4). It is most suitable for grade 3 haemorrhoids in the absence of rectal mucosal prolapse and when the external haemorrhoidal component is clear.

However, surgical treatments such as the above are relatively cumbersome, painful and require post-operative monitoring before normal urination can be resumed. Moreover, such interventions require a modulated time off work depending on the findings of the first postoperative visit and the type of professional activity. The average length of time off is one to two weeks after a haemorrhoidopexy and three to four weeks after a haemorrhoidectomy.

There is therefore a need today to develop alternative and effective solutions enabling action to be taken before surgery. Many compositions for the treatment of haemorrhoidal disease have been proposed to date.

The HEMAPRO™ composition is a cream for haemorrhoids based on a formula that contains a plant complex formed of goldenrod, lemon and butcher's broom which are known for their oedema reducing, soothing and blood circulation stimulating, and tonic properties. Together with the flavonoids and tannins of the other natural components, this composition improves the blood circulation and contributes to the healing of wounds.

DIEPHARMEX™ Laboratories have also designed an anti-haemorrhoidal gel: MICRO H™. This gel is packaged in single 5 ml doses and lines the mucous membranes of the anal canal with a protective and soothing gel, promoting natural healing.

SEDORRHOIDE™ CRISIS HAEMORRHOID rectal cream is an anti-inflammatory and a local anaesthetic. This medicine is recommended for certain anal pathologies accompanied by pain and/or itching and in particular for a haemorrhoidal crisis.

Document EP0391905 describes a topical composition based on sucrose octasulphate aluminium salt useful for the prevention and treatment of haemorrhoidal disease in addition to its healing action.

Document FR3026948 describes a topical cosmetic and/or dermatological composition, in particular useful for preventing or treating haemorrhoidal disease comprising, in a physiologically acceptable carrier, at least an effective amount of a synthetic polysulphated oligosaccharide potassium salt having 1 to 4 glycose units.

However, the use of existing compositions is not without limitations and controversy regarding their effectiveness.

The present invention relates to a composition in a form suitable for topical herbal administration, as well as to a process for its manufacture. The invention also relates to such a composition for use in the treatment or prevention of symptoms in dermatological or gastroenterological conditions, particularly proctological conditions such as haemorrhoidal disease.

In general, there is a need to search for new compositions with improved soothing and calming properties for the treatment or prevention of symptoms in dermatological or gastroenterological conditions, in particular proctological conditions such as haemorrhoidal disease. There is therefore a need to develop such new compositions as well as alternative processes for obtaining such compositions comprising plant extracts with improved soothing and calming properties.

In view of the above, one problem which the present invention proposes to solve is in particular to develop compositions which are easy and rapid to apply, simple to use, and which present an improved effectiveness (over time, both quantitatively and qualitatively) while advantageously limiting the impact on the environment.

The solution to this problem first relates to a composition in a form suitable for topical administration comprising, in a cosmetically and/or pharmaceutically acceptable vehicle, at least one nettle extract (Urtica), characterised in that it further comprises a mixture of at least five of the following twenty-three plant extracts:
marigold (Calendula);
dandelion (Taraxacum);
elderberry (Sambucus);
rosemary (Rosmarinus officinalis);
sage (Salvia officinalis);
wormwood (Artemisia absinthium L.);
burdock (Arctium lappa);
wild thyme (Thymus serpyllum);
willow (Salix);
ribwort plantain (Plantago lanceolata);
sweet flag (Acorus calamus);
aloe (Asphodeloideae);
blackberry (Morus);
cleavers (Galium aparine L.);
yellow gentian (Gentiana lutea);
angelica (Angelica archangelica);
yarrow (Achillea millefolium L.);
basil (Ocimum basilicum);
ground ivy (Glechoma hederacea);
comfrey (Symphytum officinale);
ivy (Hedera helix L.);
lemon balm (Melissa officinalis); or
common walnut (Juglans regia).

Secondly, it relates to a process for preparing a composition according to the invention, characterised in that it comprises the following steps of:
preparing the following plants or parts of plants (flowers, flowering tops, aerial parts, leaves, stems, buds, roots, bulbs, rhizomes, bark, seeds, fruits, stones, berries, sap, nothing, latex and/or thallus): marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), nettle (Urtica), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), common walnut (Juglans regia), and possibly angelica (Angelica archangelica);
heating and melting the lipophilic phase;
incorporating the various plants or parts of plants into said liquid lipophilic phase;
stirring and mixing the resulting mixture for a minimum of one hour;
filtering the mixture through a sieve with a mesh size of 0.2 mm or less, so as to remove the solid part of the mixture;
heating the filtrate thus obtained;
possibly adding yellow gentian brandy;
boiling the mixture;
removing from the heat and possibly adding white wax
cooling and recovering the resulting composition.

Thirdly, it relates to a composition according to the invention, for use in the prevention and/or treatment of symptoms in the case of dermatological disorders, in particular in humans.

Finally, the fourth object is a composition according to the invention, to prevent and treat the symptoms of haemorrhoidal disease.

The invention and the advantages resulting therefrom will be better understood by reading the following description and non-limiting embodiments.

The Applicant was able to demonstrate that the composition of the invention made it possible to increase the effectiveness of compositions to treat and prevent dermatological or gastroenterological disorders, in particular proctological disorders such as haemorrhoidal disease.

More particularly, the Applicant has been able to demonstrate that the composition according to the invention comprising at least one nettle extract (Urtica) and at least one mixture of at least five other plant extracts, applied topically, has in particular one or more of the following advantages:
an anti-inflammatory effect;
a healing effect;
a soothing effect on burns;
a soothing effect on itching;
a preventive effect on haemorrhoidal disease;
a curative effect on haemorrhoidal disease.

This increase in effectiveness allows for improved treatment or prevention of dermatological or gastroenterological conditions.

Furthermore, the pharmaceutical composition developed by the Applicant is stable and well tolerated.

Advantageously, some compositions according to the invention allow a prolonged release of the active ingredient(s). This results in a longer duration of action and a better tolerance profile.

In this description, unless otherwise specified, it is understood that, when an interval is given, it includes the upper and lower limits of said interval. Similarly, when referring to a compound, it includes any pharmaceutically acceptable form and in particular isomers, salts, solvates and polymorphs. In particular, if a compound has optical activity, the referenced compound will include the racemic mixture of the different isomers.

The invention relates to a pharmaceutical or cosmetic composition. It is presented in a form suitable for topical application, particularly adapted to its place of administration (skin, scalp, hair, etc.), preferably skin lesions or haemorrhoids.

The composition comprises, in a cosmetically and/or pharmaceutically acceptable vehicle, at least one nettle extract (Urtica).

“Cosmetically and/or pharmaceutically acceptable vehicle” means a non-toxic carrier that can be applied to at least one keratinous material of human beings. The cosmetic and/or dermatological composition is advantageously a composition for topical application, comprising a cosmetically or dermatologically acceptable medium, i.e. one which has a pleasant colour, odour and feel and which does not generate unacceptable discomfort (tingling, tightness, redness), likely to deter consumers from using this composition.

The composition according to the invention further comprises a mixture of at least five of the following twenty-three plant extracts:
wormwood (Artemisia absinthium L.);
sweet flag (Acorus calamus);
yarrow (Achillea millefolium L.);
aloe (Asphodeloideae);
angelica (Angelica archangelica);
burdock (Arctium lappa);
basil (Ocimum basilicum);
comfrey (Symphytum officinale);
cleavers (Galium aparine L.);
yellow gentian (Gentiana lutea);
ground ivy (Glechoma hederacea);
ivy (Hedera helix L.);
lemon balm (Melissa officinalis);
blackberry (Morus);
common walnut (Juglans regia).
dandelion (Taraxacum);
ribwort plantain (Plantago lanceolata);
rosemary (Rosmarinus officinalis);
sage (Salvia officinalis);
willow (Salix);
wild thyme (Thymus serpyllum);
marigold (Calendula); or
elderberry (Sambucus);

The Applicant was able to demonstrate that such a composition was particularly effective to treat and prevent dermatological or gastroenterological conditions, including proctological conditions such as haemorrhoidal disease.

When the composition comprises five of the twenty-three plant extracts listed above, it is preferably the following mixtures:
marigold, dandelion, elderberry, rosemary, sage;
dandelion, elderberry, rosemary, sage, wormwood;
elderberry, rosemary, sage, burdock, wild thyme;
rosemary, sage, burdock, wild thyme, willow;
sage, burdock, wild thyme, willow, ribwort plantain;

Preferably, the composition according to the invention comprises more than five of the twenty-three plant extracts listed above, preferably ten, twelve, fifteen or twenty plant extracts.

Among the ten preferred plant extracts are:
marigold (Calendula);
dandelion (Taraxacum);
elderberry (Sambucus);
rosemary (Rosmarinus officinalis);
sage (Salvia officinalis);
wormwood (Artemisia absinthium L.);
burdock (Arctium lappa);
wild thyme (Thymus serpyllum);
willow (Salix); and
ribwort plantain (Plantago lanceolata).

Among the twelve preferred plant extracts are:
marigold (Calendula);
dandelion (Taraxacum);
elderberry (Sambucus);
rosemary (Rosmarinus officinalis);
sage (Salvia officinalis);
wormwood (Artemisia absinthium L.);
burdock (Arctium lappa);
wild thyme (Thymus serpyllum);
willow (Salix);
ribwort plantain (Plantago lanceolata);
sweet flag (Acorus calamus); and
aloe (Asphodeloideae).

Among the fifteen preferred plant extracts are:
marigold (Calendula);
dandelion (Taraxacum);
elderberry (Sambucus);
rosemary (Rosmarinus officinalis);
sage (Salvia officinalis);
wormwood (Artemisia absinthium L.);
burdock (Arctium lappa);
wild thyme (Thymus serpyllum);
willow (Salix);
ribwort plantain (Plantago lanceolata);
sweet flag (Acorus calamus);
aloe (Asphodeloideae);
blackberry (Morus);
cleavers (Galium aparine L.); and
yellow gentian (Gentiana lutea);

Among the twenty preferred plant extracts are:
marigold (Calendula);
dandelion (Taraxacum);
elderberry (Sambucus);
rosemary (Rosmarinus officinalis);
sage (Salvia officinalis);
wormwood (Artemisia absinthium L.);
burdock (Arctium lappa);
wild thyme (Thymus serpyllum);
willow (Salix);
ribwort plantain (Plantago lanceolata);
sweet flag (Acorus calamus);
aloe (Asphodeloideae);
blackberry (Morus);
cleavers (Galium aparine L.);
yellow gentian (Gentiana lutea);
yarrow (Achillea millefolium L.);
basil (Ocimum basilicum);
ground ivy (Glechoma hederacea);
comfrey (Symphytum officinale); and
ivy (Hedera helix L.);

Preferably, the composition according to the invention comprises a nettle extract (Urtica) and the following set of plant extracts:

marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), common walnut (Juglans regia) and possibly angelica (Angelica archangelica).

The various extracts are preferably dispersed in a lipophilic phase composed of oils, fats and/or waxes of vegetable, animal, mineral or synthetic origin.

Non-limiting examples of vegetable oils that can be used include olive oil, almond oil, peanut oil, soybean oil and sunflower oil, either alone or in combination. Preferably, the vegetable oil is olive oil.

Non-limiting examples of usable vegetable fats include shea butter, cocoa butter and mango butter, either alone or in a mixture. Preferably, the vegetable fat is shea butter.

Non-limiting examples of vegetable oils that can be used include carnauba wax (cactus), soy wax, jojoba wax and candela wax (palm), either alone or in mixture. Preferably, the vegetable wax is soy wax.

Non-limiting examples of animal oils that can be used include whale oil, sperm whale oil, shark liver oil, mink oil and cod oil, either alone or in combination. Preferably, the animal oil is cod oil.

Non-limiting examples of usable animal fats that can be used include lanolin, seal milk, whale milk, lard and tallow, either alone or in combination. Preferably, the animal fat is lard.

Non-limiting examples of usable animal fats that can be used include beeswax or spermaceti, either alone or in combination. Preferably, the animal wax is beeswax.

Non-limiting examples of mineral oils, fats or waxes that can be used include petroleum jelly and paraffin, either alone or in combination.

Non-limiting examples of synthetic oils, fats or waxes that can be used include silicone oil or silicone wax, fatty esters and fatty alcohols, either alone or in combination.

The lipophilic phase is preferably an animal fatty phase, more preferably lard.

According to the invention, the fatty phase represents at least 10% by weight of the total weight of the composition (w/w).

Preferably, the fatty phase represents between 10% and 99% by weight of the total weight of the composition (w/w). Even more preferably, the fatty phase represents between 50% and 90% by weight of the total weight of the composition (w/w). Indeed, for such a range of fatty phase proportions, a good compromise is observed between a stable emulsion containing enough fatty phase for dermatological application (especially for dry skin) and good tissue tolerance.

A high fatty phase content is particularly interesting for dermatological compositions intended for skin care and especially for dry skin.

The composition according to the invention may further comprise one or more additives compatible with cosmetic and/or dermatological use.

Of course, the person skilled in the art shall take care to choose the adjuvant(s) to be added to the compositions according to the invention, as well as their concentration, so that the advantageous properties intrinsically attached to the compositions according to the invention are not, or substantially not, altered by the addition envisaged.

In particular, these adjuvants must not impair the properties of the composition according to the invention, i.e. in particular the absence of skin irritability.

According to an advantageous embodiment of the invention, the pharmaceutical composition according to the invention may contain other additional ingredients such as an adjuvant, an anti-oxidant, a chelating agent, a surfactant, a foaming agent, a wetting agent, an emulsifying agent, a viscosifier, a buffering agent or a preservative, preferably a preservative.

In a further advantageous embodiment, the pharmaceutical composition according to the invention may contain a permeation or penetration enhancer.

A permeation or penetration enhancer is defined as a compound that significantly improves the skin penetration of the active ingredient according to the invention.

Illustrative examples of permeation enhancers include oleic acid, oleic alcohol, ethoxydiglycol, laurocapram, alkanecarboxylic acid, dimethylsulfoxide, polar lipids or N-methyl-2-pyrrolidone.

According to an advantageous embodiment, the pharmaceutical composition according to the invention may contain a hydrotropic agent disrupting the structure of the stratum corneum and thereby further increasing penetration through the surface layer of the skin.

An illustrative example of a hydrotropic agent is sodium xylene sulphonate, isopropyl alcohol, and preferably propylene glycol.

According to a particular embodiment of the invention, the composition further comprises at least one pharmaceutically active ingredient selected from soothing agents, agents promoting healing and/or restructuring of the skin, anti-allergic agents, anti-bacterial agents, depigmenting agents, moisturising agents, keratolytic agents, analgesics, anaesthetics, pain relievers_{[VIW1]}, antibiotics, antifungals, painkillers, anti-inflammatories, antiparasitic agents, antipruritic agents, antiseptics, antiviral agents, anti-acne agents, corticosteroids, vasoconstrictors, venotonics and/or a mixture thereof.

Non-limiting examples of a pharmaceutically active ingredient that can be used according to the invention include, without this list being exhaustive: acetaminophen, fluocinolone acetonide, acetylsalicylic acid, acitretin, clavulanic acid, azelaic acid, acyclovir, adapalene, alclometasone, alpha-tocopherol, amcinonide, amorolfine, amphotericin B, tetracycline, benzoyl peroxide, betamethasone, calcipotriol, calcitriol, ciclopirox, clindamycin, codeine, crisaborole, clobetasol, crotamiton, cyproheptadine, dapsone, desonide, diosmin, diclofenac, diflucortolone, difluprednate, dioxyanthranol, econazole, efinaconazole, erythromycin, estradiol, etretinate, fluticasone, fusidic acid, mometasone, glycolic acid, glycyrrhetinic acid, halobetasol, hydrocortisone, hydroquinone, ibuprofen, imiquimod, isotretinoin, ivermectin, ketoconazole, kojic acid, lactic acid, lidocaine, malic acid, mequinol, methoxsalen, metronidazole, miconazole, minocycline, minoxidil, morphine, octopirox, oxymetazoline, paracetamol, penicillins (amoxycilin), pilocaine, pyridoxine, progesterone, retinol, pimecrolimus, resiquimod, rucinol, tacrolimus, tazarotene, terbinafine, tetracaine thenaldine, travopost, tretinoin, trimeprazine, trifarotene, troxerutin, zinc pyrithione, as well as salts or derivatives of these active ingredients, either alone or in combination.

The compositions according to the invention may be presented in any galenic form normally used by the person skilled in the art.

Preferably, the compositions according to the invention are intended for topical administration and are in the form of a solution, a dispersion, an emulsion of liquid or semi-liquid consistency, a suspension, an aqueous or lipophilic gel, a microemulsion, a microcapsule, a vesicular dispersion, a dressing or an impregnated support article.

Particularly advantageously, the composition according to the invention is in the form of an ointment.

According to a preferred embodiment of the invention, the composition is in the form of an ointment comprising:
a lipophilic phase comprising all the extracts of the following plants: marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), nettle (Urtica), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), common walnut (Juglans regia), and possibly angelica (Angelica archangelica); and
a hydrophilic phase comprising yellow gentian (Gentiana lutea) brandy.

The invention also relates to a process for preparing a composition according to one of the preceding claims.

Advantageously, the preparation process comprises the following steps:
preparing the following plants or parts of plants (flowers, flowering tops, aerial parts, leaves, stems, buds, roots, bulbs, rhizomes, bark, seeds, fruits, stones, berries, sap, nothing, latex and/or thallus): marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), nettle (Urtica), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), common walnut (Juglans regia), and possibly angelica (Angelica archangelica);
heating and melting the lipophilic phase;
incorporating the various plants or parts of plants into said liquid lipophilic phase;
stirring and mixing the mixture thus obtained for a period of at least one hour; preferably at least two hours; more preferably between two and three hours;
filtering the mixture through a sieve with a mesh size of 0.2 mm or less, so as to remove the solid part of the mixture;
heating the filtrate thus obtained;
possibly adding yellow gentian brandy;
boiling the mixture;
removing from the heat and possibly adding white wax
   cooling and recovering the resulting composition.

The effective amounts of plants that can be used can be adapted according to the end use of the product.

“Effective amount” is meant, in the sense of the present invention, an amount sufficient to obtain the expected effect.

Preferably, the amount of plant or plant extract incorporated in the liquid lipophilic phase is between:
0.1% and 30%, preferably between 5% and 25% and even more preferably between 10% and 20% for nettle;
0% and 15%, preferably between 1% and 10% and even more preferably between 1.5% and 3% for marigold;
0% and 5%, preferably between 0.1% and 2% and even more preferably between 0.4% and 1% for dandelion;
0% and 10%, preferably between 1% and 7% and even more preferably between 2% and 6% for elderberry;
0% and 10%, preferably between 1% and 5% and even more preferably between 2% and 4% for rosemary;
0% and 10%, preferably between 0.5% and 5% and even more preferably between 1% and 3% for sage;
0% and 2%, preferably between 0.05% and 1% and even more preferably between 0.1% and 0.5% for wormwood;
0% and 5%, preferably between 0.3% and 3% and even more preferably between 0.5% and 1.5% for burdock;
0% and 3%, preferably between 0.1% and 2% and even more preferably between 0.3% and 1% for wild thyme;
0% and 5%, preferably between 1% and 3% and even more preferably between 1.5% and 2.6% for willow;
0% and 3%, preferably between 0.2% and 2.5% and even more preferably between 0.4% and 2% for ribwort plantain;
0% and 5%, preferably between 1% and 3% and even more preferably between 1.5% and 2% for sweet flag;
0% and 2%, preferably between 0.2% and 1% and even more preferably between 0.3% and 0.7% for aloe;
0% and 2%, preferably between 0.1% and 1% and even more preferably between 0.2% and 0.8% for blackberry;
0% and 3%, preferably between 0.4% and 1.5% and even more preferably between 0.6% and 1.2% for cleavers;
0% and 10%, preferably between 1% and 5% and even more preferably between 1.5% and 3% for yellow gentian;
0% and 10%, preferably between 2% and 8% and even more preferably between 3% and 6% for yarrow;
0% and 1%, preferably between 0.01% and 0.5% and even more preferably between 0.05% and 0.3% for basil;
0% and 2%, preferably between 0.05% and 1% and even more preferably between 0.1% and 0.7% for ground ivy;
0% and 3%, preferably between 1% and 2.5% and even more preferably between 1.2% and 2% for comfrey;
0% and 2%, preferably between 0.05% and 1% and even more preferably between 0.1% and 0.4% for ivy;
0% and 6%, preferably between 1.5% and 5% and even more preferably between 2% and 4% for lemon balm;
0% to 2%, preferably between 0.1% and 1% and more preferably between 0.3% and 0.6% for common walnut; and
0% and 2%, preferably between 0% and 1% and even more preferably between 0% and 0.2% for angelica.

The pharmaceutical composition which is the subject matter of the invention is particularly suitable for use in the prevention and/or treatment of dermatological disorders, especially in humans.

More particularly, the pharmaceutical composition which is the subject matter of the invention is suitable for use in the prevention and/or treatment of, in particular, human dermatological disorders.

Examples of dermatological conditions include:
1) dermatological conditions linked to a keratinisation disorder concerning cellular differentiation and proliferation, in particular to treat acne vulgaris, acne comedonica, polymorphs, rosacea, nodulocystic acne, conglobata, senile acne, secondary acne such as solar, drug-induced or professional acne;
2) keratinisation disorders, including ichthyosis, ichthyosiform conditions, lamellar ichthyosis, Darier's disease, palmoplantar keratoderma, leukoplakia, Pityriasis rubra pilaris and leukoplakiform conditions, cutaneous or mucosal (oral) lichen;
3) dermatological diseases with an inflammatory immuno-allergic component, with or without a disorder of cell proliferation, and in particular all forms of psoriasis, whether cutaneous, mucous or nail, and even psoriatic rheumatism, or atopic dermatitis and the various forms of eczema;
4) skin disorders due to exposure to UV radiation as well as to repair or combat skin ageing, whether photo-induced or chronological, or to reduce pigmentation and actinic keratoses, or any pathologies associated with chronological or actinic ageing, such as xerosis, pigmentation and wrinkles;
5) any condition related to benign dermal or epidermal proliferations, whether or not of viral origin, such as verrucae vulgaris, flat warts, molluscum contagiosum and epidermodysplasia verruciformis, oral or florid papillomatosis;
6) dermatological disorders such as immune dermatoses like lupus erythematosus, immunobullous diseases and collagen diseases like scleroderma;
7) the stigma of epidermal and/or dermal atrophy induced by local or systemic corticosteroids, or any other form of skin atrophy,
8) wound healing disorders, or to prevent or repair stretch marks, or to promote healing,

In other words, the invention relates to a pharmaceutical composition according to the invention for use as a medicine to treat dermatological conditions, in particular human dermatological conditions, as previously defined.

Finally, as is apparent from the examples below, the composition which is the subject matter of the invention is particularly suitable for use in the prevention and/or treatment of gastroenterological disorders, especially human disorders, particularly proctological disorders such as the prevention or treatment of haemorrhoidal disease.

“Prevention or treatment” is meant both any action on the symptoms of haemorrhoids, i.e. concerning the aesthetic aspect of said haemorrhoids, and any action on the cause, i.e. any action on the longitudinal muscularis of the rectum forming the Parks ligament and/or on the opening of arteriovenous shunts, but also any action on the consequences of said haemorrhoids, i.e. the scars resulting from said haemorrhoids.

The composition is also suitable for relieving burns, aiding healing, relieving haemorrhoidal attacks and has an anti-inflammatory effect.

The present invention will now be illustrated with the following examples.

### EXAMPLE 1: LIST OF THE DIFFERENT PLANTS OR PARTS OF PLANTS THAT CAN BE USED TO MANUFACTURE A COMPOSITION ACCORDING TO THE INVENTION

The various ingredients listed below are advantageously used in quantities to prepare approximately 5 kg of finished product:

1. Marigold or Calendula (Calendula officinalis)

To prepare the composition according to the invention, which is advantageously an ointment, 200 g of fresh marigold leaves are advantageously used. Fresh marigold leaves can be replaced by dried marigold leaves. Marigold is particularly useful for the treatment of wounds and cuts.

2. Dandelion (Taraxacum, Especially Taraxacum albidum)

To prepare the ointment according to the invention, 50 g to 100 g of finely chopped fresh dandelion roots are advantageously used.

Dandelion is particularly useful for treating wounds and cuts and promotes good breathing.

3. Elderberry (Sambucus)

To prepare the ointment according to the invention, it is advantageous to use about 200 g of fresh, non-compacted elderflowers and about 50 g of fine (green and soft) elderberry.

Fresh elderberry flowers can be replaced by dried elderberry flowers. It is preferable to use three times as much.

Elderberry flowers are particularly useful for wound treatment and skin healing.

4. Rosemary (Rosmarinus officinalis)

To prepare the ointment according to the invention, about 200 g of fresh rosemary needles are advantageously used. Fresh rosemary needles can be replaced by dried rosemary needles. It is preferable to use twice as much.

5. Sage or Common Sage (Salvia officinalis)

To prepare the ointment according to the invention, about 100 g to 150 g of fresh sage leaves are advantageously used. Fresh sage leaves can be replaced by dried sage leaves. It is preferable to use twice as much.

Sage is particularly useful for the lungs, breathing and the treatment of inflammation.

6. Wormwood or Grand Wormwood (Artemisia absinthium L.)

To prepare the ointment according to the invention, fresh wormwood leaves from two branches of approximately 30 to 40 cm in length are advantageously used. It is possible to replace fresh wormwood leaves with dried ones. It is preferable to use the same amount (fresh or dry). Wormwood is particularly useful for its antiseptic properties.

7. Burdock (Arctium lappa)

To prepare the ointment according to the invention, fresh medium-sized burdock leaves are advantageously used. Fresh burdock leaves can be replaced by dried burdock leaves. It is preferable to use three times as much. Burdock leaves are particularly useful for the treatment of wounds and for the joints. To prepare the ointment according to the invention, it is also recommended to integrate two fresh burdock roots of medium size, from young plants and finely chopped. Burdock roots are particularly useful for the rapid healing of wounds.

8. Wild Thyme (Thymus serpyllum)

To prepare the ointment according to the invention, about 40 g to 50 g of fresh wild thyme leaves are advantageously used. Fresh wild thyme leaves can be replaced by dried wild thyme leaves. It is preferable to use twice as much.

9. Willow (Salix)

To prepare the ointment according to the invention, it is advantageous to use approximately 200 g of fresh, chopped curly willow leaves. The leaves are generally available all year round.

10. Ribwort Plantain (Plantago lanceolata)

To prepare the ointment according to the invention, about 50 g to 100 g of fresh juicy young leaves, i.e. about 40 ribwort plantain leaves, more preferably a mixture of male and female plants, are advantageously used. Fresh ribwort plantain leaves can be replaced by dried ribwort plantain leaves. It is preferable to use twice as much. Ribwort plantain leaves are useful in the treatment of many ailments, particularly ear infections and inflammation.

11. Sweet Flag (Acorus calamus)

To prepare the ointment according to the invention, about 150 g to 200 g of fresh, finely chopped and well-cleaned roots (about two roots) of sweet flag are advantageously used. Sweet flag root is useful in the treatment of many ailments. The roots are generally available all year round.

12. Aloe (Asphodeloideae)

To prepare the ointment according to the invention, about one medium-sized bunch of small fresh aloe leaves and/or flowers is advantageously used. The leaves are generally available all year round.

13. Blackberry (Morus)

To prepare the ointment according to the invention, about 20 to 30 fresh blackberry leaves, i.e. about 30 to 40 g, are advantageously used. The fresh blackberry leaves can be replaced by dried blackberry/bramble leaves. It is preferable to use twice as much.

14. Nettle (Urtica)

To prepare the ointment according to the invention, it is advantageous to use approximately 1 kg of fresh nettle aerial parts. Fresh nettles can be replaced by dried ones. It is preferable to use twice as much. The aerial parts of the nettle are the basis of the preparation.

15. Cleavers (Galium aparine L.)

To prepare the ointment according to the invention, it is advantageous to use about two long branches of cleavers, i.e. about 60 g, coarsely chopped. It is best not to use wood but soft branches. The branches of cleavers are particularly useful for the treatment of many dermatological conditions. It is possible but difficult to use dried branches. If necessary, fresh branches should be replaced by dried ones. It is preferable to use twice as much.

16. Yellow Gentian (Gentiana lutea)

To prepare the ointment according to the invention, about 200 g of clean, fresh yellow gentian roots, about 1 year old, are advantageously used. The roots are preferably finely chopped and fresh. Yellow gentian root is useful in the treatment of many ailments. In particular, they have a protective effect on the blood vessels and the liver and help to clean the body. They also have an antiseptic effect.

17. True Angelica, Archangelica or Garden Angelica (Angelica archangelica)

To prepare the ointment according to the invention, about 20 g of angelica flowers and/or roots are advantageously used. The addition of angelica to the preparation is optional.

18. Yarrow (Achillea millefolium L.)

To prepare the ointment according to the invention, about 300 g of fresh yarrow flowers are advantageously used. Fresh yarrow flowers can be replaced by dried yarrow flowers. It is preferable to use twice as much. Yarrow leaves are particularly useful for treating wounds and lung diseases.

19. Basil (Ocimum basilicum)

To prepare the ointment according to the invention, it is advantageous to use about 10 g of fresh basil leaves, i.e. about 15 leaves.

The leaves are generally available all year round.

20. Ground Ivy (Glechoma hederacea)

To prepare the ointment according to the invention, it is advantageous to use about 20 g to 30 g of fresh ground ivy, in pieces of about 15 cm from the top, with a flower, chopped into pieces. Fresh ground ivy can be replaced by dried ground ivy. It is preferable to use twice as much.

21. Comfrey (Symphytum officinale)

To prepare the ointment according to the invention, it is advantageous to use approximately 150 g of fresh comfrey roots, unpeeled, well washed, cut into small pieces. Fresh comfrey root is particularly useful for strengthening bones.

22. Ivy or Climbing Ivy (Hedera helix L.)

To prepare the ointment according to the invention, it is advantageous to use about 30 g of fresh ivy leaves, i.e. about 20 small young leaves.

23. Lemon Balm (Melissa officinalis)

To prepare the ointment according to the invention, about 200 g of fresh lemon balm leaves (tips) are advantageously used. Fresh lemon balm leaves can be replaced by dried lemon balm leaves. It is preferable to use twice as much.

24. Common Walnut Leaf

To prepare the ointment according to the invention, it is advantageous to use about 50 g of young common walnut leaves (tips). Fresh leaves from the common walnut tree (Juglans regia), about 10 to 12 leaves, cut into small pieces, can be substituted.

In general, if using ingredients in dried form, double the quantities used and do not use ingredients that have been dried for more than two years.

### EXAMPLE 2: PRODUCTION PROCESS FOR A COMPOSITION ACCORDING TO THE INVENTION IN A LARD BASE

The base of the composition is pork fat (lard) and nettles. The different stages of preparation are detailed below:
Preparing all or some of the ingredients described in example 1 above;
heating and melting about 5 kg of lipophilic phase which is the lard;
incorporating the various plants or parts of plants into said liquid lipophilic phase;
stirring and mixing the resulting mixture for two to three hours over low heat;
filtering the mixture, for example through a sieve with a mesh size of 0.2 mm or less, or through gauze placed on a sieve;
removing the solid part of the mixture;
heating the filtrate thus obtained;
adding about 50 ml of yellow gentian brandy;
boiling the mixture;
removing from the heat and adding about 1 kg of beeswax;
cooling and recovering the resulting composition; and
distributing the composition in suitable containers.

### EXAMPLE 3: EFFICACY TEST OF THE COMPOSITION ACCORDING TO THE INVENTION

The herbal extract composition according to the invention was tested on a panel of three people with symptoms of haemorrhoidal disease. These symptoms include itching, burning and irritation, and haemorrhoidal episodes

After using the composition based on plant extracts, these people noticed a clear reduction in the symptoms associated with haemorrhoidal disease and an improvement in their comfort.

The main effects were soothing of itching, soothing of burns, healing and soothing of haemorrhoidal episodes.

These effects were observed by all the people tested, regardless of the first day of application of the plant extract-based composition.

However, the effects are most effective and quickest if the first day of application occurs as soon as the first symptoms of haemorrhoidal disease appear.

1. Composition in a form suitable for topical administration comprising, in a cosmetically and/or pharmaceutically acceptable vehicle, at least one nettle extract (Urtica), characterised in that it further comprises a mixture of at least five of the following twenty-three plant extracts:
marigold (Calendula);
dandelion (Taraxacum);
elderberry (Sambucus);
rosemary (Rosmarinus officinalis);
sage (Salvia officinalis);
wormwood (Artemisia absinthium L.);
burdock (Arctium lappa);
wild thyme (Thymus serpyllum);
willow (Salix);
ribwort plantain (Plantago lanceolata);
sweet flag (Acorus calamus);
aloe (Asphodeloideae);
blackberry (Morus);
cleavers (Galium aparine L.);
yellow gentian (Gentiana lutea);
angelica (Angelica archangelica);
yarrow (Achillea millefolium L.);
basil (Ocimum basilicum);
ground ivy (Glechoma hederacea);
comfrey (Symphytum officinale);
ivy (Hedera helix L.);
lemon balm (Melissa officinalis); or
common walnut (Juglans regia).

2. The composition according to claim 1, wherein the mixture of at least five plant extracts comprises: marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), and common walnut (Juglans regia).

3. The composition according to claim 1, wherein the extracts are preferably dispersed in a lipophilic phase.

4. The composition according to claim 3, wherein the lipophilic phase is an animal fatty phase.

5. The composition according to claim 1, further comprising at least one pharmaceutically active ingredient selected from the group consisting of soothing agents, agents promoting healing and/or restructuring of the skin, anti-allergic agents, anti-bacterial agents, depigmenting agents, moisturizing agents, keratolytic agents, analgesics, anaesthetics, pain relievers, antibiotics, antifungals, painkillers, anti-inflammatories, antiparasitic agents, antipruritic agents, antiseptics, antiviral agents, anti-acne agents, corticosteroids, vasoconstrictors, venotonics and mixtures thereof.

6. The composition according to claim 1, wherein the composition is in the form of a solution, a dispersion, an emulsion of liquid or semi-liquid consistency, a suspension, an aqueous gel, a lipophilic gel, a microemulsion, a microcapsule, a vesicular dispersion, a dressing, or an impregnated support article.

7. The composition according to claim 6, wherein the composition is in the form of an ointment comprising:
a lipophilic phase comprising all the extracts of the following plants: marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), nettle (Urtica), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), and common walnut (Juglans regia); and
a hydrophilic phase comprising yellow gentian (Gentiana lutea) brandy.

8. Preparation process for a composition according to claim 7, the method comprising:
preparing the following plants or parts of plants (flowers, flowering tops, aerial parts, leaves, stems, buds, roots, bulbs, rhizomes, bark, seeds, fruits, stones, berries, sap, nothing, latex and/or thallus): marigold (Calendula), dandelion (Taraxacum), elderberry (Sambucus), rosemary (Rosmarinus officinalis), sage (Salvia officinalis), wormwood (Artemisia absinthium L.), burdock (Arctium lappa), wild thyme (Thymus serpyllum), willow (Salix), ribwort plantain (Plantago lanceolata), sweet flag (Acorus calamus), aloe (Asphodeloideae), blackberry (Morus), nettle (Urtica), cleavers (Galium aparine L.), yellow gentian (Gentiana lutea), yarrow (Achillea millefolium L.), basil (Ocimum basilicum), ground ivy (Glechoma hederacea), comfrey (Symphytum officinale), ivy (Hedera helix L.), lemon balm (Melissa officinalis), and common walnut (Juglans regia);
heating and melting the lipophilic phase;
incorporating the various plants or parts of plants into said liquid lipophilic phase;
stirring and mixing the resulting mixture for a minimum of one hour;
filtering the mixture to obtain a filtrate;
heating the filtrate; and
cooling and recovering the resulting composition.

9. (canceled)

10. (canceled)

11. The method of claim 8, wherein filtering comprises passing the mixture through a sieve with a mesh size of less than or equal to 0.2 mm, thereby removing solids from the mixture.

12. The method of claim 8, comprising adding yellow gentian brandy to the filtrate to form a mixture, and boiling the mixture.

13. The method of claim 12, comprising cooling the mixture and subsequently adding beeswax.

14. The composition according to claim 2, wherein the lipophilic phase comprises a vegetable oil, a vegetable fat, a vegetable wax, an animal fat, an animal, fat, an animal wax, a mineral oil, a mineral fat, a mineral wax, a synthetic oil, a synthetic fat, a synthetic wax, or combinations thereof.

15. The composition according to claim 4, wherein the animal fatty phase comprises lard.

16. The composition according to claim 6, wherein the composition is in the form of an ointment.

17. A treatment method for dermatological disorders, the method comprising:
identifying a patient having a dermatological disorder and a site in need of treatment;
applying a composition according to claim 1 to the site.

18. A treatment method for haemorrhoidal disease, the method comprising:
identifying a patient having a haemorrhoidal disease in need of treatment;
applying a composition according to claim 1 to a haemorrhoidal site of the patient.
